(19) United States
(12) Patent Application Publication
(10) Pub. No.: US 2009/0092657 A1
(43) Pub. Date: Apr. 9, 2009

(54) Amino acid combinant omega oil aphrodisiac

(76) Inventors: Douglas Burke, Newport Beach, CA (US); Laila S. Nabulsi, Costa Mesa, CA (US)

Correspondence Address: Douglas Burke, 2507 Port Whitby, Newport Beach, CA, 92660, US

(21) Appl. No.: 11/973,601

(22) Filed: Oct. 9, 2007

(51) Int. Cl. A61K 36/55; A61K 31/198; A61K 31/405; A61P 15/00; A61K 47/46; A61K 36/18

(52) U.S. Cl. 424/439; 424/768; 424/776; 514/412; 514/567

(57) ABSTRACT

A method of enhancing the libido in an adult human comprising the administering of a composition, comprising an effective amount of the amino acids tryptophan and phenylalanine and an effective amount of omega oils.

### FIELD OF THE INVENTION

The invention relates to a new combination of substances comprising amino acids, which when combined with omega oils and a chocolate blend, constitute a powerful aphrodisiac.

### BACKGROUND OF THE INVENTION

Tryptophan is an essential amino acid involved in human nutrition. It is one of the 20 amino acids encoded by the genetic code (as codon UGG). Only the L-stereoisomer appears in mammalian protein, but the D-stereoisomer is occasionally found in natural materials (for example, the marine venom peptide contryphan). A distinguishing structural characteristic of tryptophan is that it contains an indole functional group.

The isolation of tryptophan was first reported by Sir Frederick Hopkins in 1901, Hopkins F G, Cole S W (1901). “A contribution to the chemistry of proteids: Part I. A preliminary study of a hitherto undescribed product of tryptic digestion”. J. Physiol. (Lond.) 27 (4-5): 418-28. It has been obtained by hydrolysis of casein. From 600 g of crude casein, one obtains ca. 4-8 grams of tryptophan.

For many organisms including humans, tryptophan is an essential amino acid. This means that it cannot be synthesized by the organism and therefore must be part of its diet. The principle function of amino acids, including tryptophan, is to act as building blocks in protein biosynthesis. In addition, tryptophan functions as a biochemical precursor for the following:
Serotonin (a neurotransmitter, via tryptophan hydroxylase. Serotonin, in turn, can be converted to melatonin (a neurohormone), via N-acetyltransferase and 5-hydroxyindole-O-methyltransferase.
Niacin via kynurenine and quinolinic acids as key biosynthetic intermediates.

In organisms that synthesize tryptophan, high levels of this amino acid activate a repressor protein, which in turn binds to the trp operon. Binding of this repressor to its operon prevents transcription of downstream DNA that codes for the enzymes involved in the biosynthesis of tryptophan. Hence high levels of tryptophan prevent additional tryptophan synthesis through a negative feedback loop. Conversely, if the cell's tryptophan level drops, transcription of the operon's genes resumes. This is one example of how gene expression responds rapidly to changes in the cell's internal and external environment.

Tryptophan, found as a component of dietary protein, is particularly plentiful in chocolate, oats, bananas, dried dates, milk, yogurt, cottage cheese, red meat, eggs, fish, poultry, sesame, chickpeas, sunflower seeds, pumpkin seeds, spirulina and peanuts. It is found in turkey at a level typical of poultry in general.

For some time, tryptophan was available in health food stores as a dietary supplement. Many people found tryptophan to be a safe and reasonably effective sleep aid, probably due to its ability to increase brain levels of serotonin (a calming neurotransmitter when present in moderate levels) and/or melatonin (a sleep-inducing hormone secreted by the pineal gland in response to darkness or low light levels).

Clinical research tended to confirm tryptophan's effectiveness as a sleep aid (Hartmann E (1982). “Effects of L-tryptophan on sleepiness and on sleep”. Journal of psychiatric research 17 (2): 107-13) (Schneider-Helmert D, Spinweber C L (1986). “Evaluation of L-tryptophan for treatment of insomnia: a review”. Psychopharmacology (Berl.) 89 (1): 1-7.), and for a growing variety of other conditions typically associated with low serotonin levels or activity in the brain such as premenstrual dysphoric disorder and seasonal affective disorder. In particular, tryptophan showed considerable promise as an antidepressant alone (Jepson T L, Ernst M E, Kelly M W (1999). “Current perspectives on the management of seasonal affective disorder”. J Am Pharm Assoc (Wash) 39 (6): 822-9.), and as an “augmenter” of antidepressant drugs (Thomson J, Rankin H, Ashcroft G W, Yates C M, McQueen J K, Cummings S W (1982). “The treatment of depression in general practice: a comparison of L-tryptophan, amitriptyline, and a combination of L-tryptophan and amitriptyline with placebo”. Psychological medicine 12 (4): 741-51). However others have questioned the reliability of these clinical trials (Levitan R D, Shen J H, Jindal R, Driver H S, Kennedy S H, Shapiro C M (2000). “Preliminary randomized double-blind placebo-controlled trial of tryptophan combined with fluoxetine to treat major depressive disorder: antidepressant and hypnotic effects”. Journal of psychiatry & neuroscience: JPN 25 (4): 337-46. PMID 11022398; Meyers S (2000). “Use of neurotransmitter precursors for treatment of depression”. Alternative medicine review: a journal of clinical therapeutic 5 (1): 64-71.

5-Hydroxytryptophan (5-HTP), a metabolite of tryptophan, has been suggested as a treatment for epilepsy and depression although clinical trials are inconclusive and lacking.

5-HTP readily crosses the blood brain barrier and in addition is rapidly decarboxylated to serotonin (5-hydroxytryptamine or 5-HT) and therefore may be useful for the treatment of depression. However serotonin has a relatively short half-life since it is rapidly metabolized by monoamine oxidase, therefore is likely to have limited efficacy. It is marketed in Europe for depression and other indications under brand names like Cincofarm and Tript-OH.

In the United States, 5-HTP does not require a prescription as it is covered under the Dietary Supplement Act. However, since the quality of dietary supplements are not regulated by the FDA, the quality of dietary and nutritional supplements tends to vary and there is no guarantee that the label accurately depicts what the bottle contains. Most health-food stores sell 5-HTP to avoid the artificially high cost of the amino acid itself.

In 1989, a large outbreak (1500 cases including at least 37 deaths) of a disabling autoimmune illness called eosinophilia-myalgia syndrome (EMS) was traced by some epidemiological studies to L-tryptophan supplied by a Japanese manufacturer, Showa Denko KK. It was further hypothesized that one or more trace impurities produced during the manufacture of tryptophan, may have been responsible for the EMS outbreak. However, many people who consumed Showa Denko L-tryptophan did not develop EMS and cases of EMS have occurred prior to and after the 1989 epidemic. Furthermore, the methodology used in the initial epidemiological studies has been criticized. An alternative explanation for the 1989 EMS outbreak is that large doses of tryptophan produce metabolites, which inhibit the normal degradation of histamine and excess histamine in turn has been proposed to cause EMS.

Most tryptophan was banned from sale in the US in 1991, and other countries followed suit. Tryptophan from one manufacturer of six, continued to be sold for manufacture of baby formulas. A Rutgers Law Journal article observed, “Political pressures have played a role in the FDA's decision to ban L-tryptophan as well as its desire to increase its regulatory power over dietary supplements.”

At the time of the ban the FDA did not know, or did not indicate, that EMS was caused by a contaminated batch, and yet even when the contamination was discovered and the purification process fixed, the FDA maintained that L-tryptophan was unsafe. In February 2001 the FDA loosened the restrictions on marketing (though not on importation), but still expressed the following concern:
“Based on the scientific evidence that is available at the present time, we cannot determine with certainty that the occurrence of EMS in susceptible persons consuming L-tryptophan supplements derives from the content of L-tryptophan, an impurity contained in the L-tryptophan, or a combination of the two in association with other, as yet unknown, external factors.”

Since 2002, L-tryptophan has been sold in the U.S. in its original form. Several high quality sources of L-tryptophan do exist, and are sold in many of the largest health food stores nationwide. Indeed, tryptophan has continued to be used in clinical and experimental studies employing human patients and subjects.

In recent years in the U.S., compounding pharmacies and some mail-order supplement retailers have begun selling tryptophan to the general public. Tryptophan has also remained on the market as a prescription drug (Tryptan), which some psychiatrists continue to prescribe, particularly as an augmenting agent for people who are unresponsive to antidepressant drugs.

It has been demonstrated in both animal models and in humans that ingestion of a meal rich in carbohydrates triggers release of insulin. Insulin in turn stimulates the uptake of large neutral branched-chain amino acids (LNAA) but not tryptophan (trp) into muscle increasing the ratio of trp to LNAA in the blood stream. The resulting increased ratio of tryptophan to large neutral amino acids in the blood reduces competition with other amino acids for the large neutral amino acid transporter protein for uptake of tryptophan across the blood-brain barrier into the central nervous system (CNS). Once inside the CNS, tryptophan is converted into serotonin in the raphe nuclei by the normal enzymatic pathway. The resultant serotonin is further metabolized into melatonin by the pineal gland. Hence, the data suggests that “feast-induced drowsiness”, and particularly, the common American post-Thanksgiving dinner drowsiness, may be the result of a heavy meal rich in carbohydrates, which via an indirect mechanism, increases the production of sleep-promoting serotonin and melatonin in the brain.

Tryptophan is not known to have any aphrodisiac effects. In fact due to its function as a sleep aid it would not be obvious to use it in an aphrodisiac formula.

Phenylalanine is an α-amino acid with the formula HO₂CCH(NH₂)CH₂C₆H₅. This essential amino acid is classified as nonpolar because of the hydrophobic nature of the benzyl side chain. The codons for L-phenylalanine are UUU and UUC. It is a white, powdery solid. L-Phenylalanine (LPA) is an electrically, neutral amino acid, one of the twenty common amino acids used to biochemically form proteins, coded for by DNA.

Phenylalanine cannot be made by animals, which have to obtain it from their diet. It is produced by plants and most microorganisms from prephenate, an intermediate on the shikimat pathway (Nelson, D. L.; Cox, M. M. “Lehninger, Principles of Biochemistry” 3rd Ed. Worth Publishing: New York, 2000. ISBN 1-57259-153-6).

L-phenylalanine can also be converted into L-tyrosine, another one of the DNA-encoded amino acids. L-tyrosine in turn is converted into L-Dopa, which is further converted into dopamine norepinephrine (noradrenaline), and epinephrine (adrenaline). (The latter three are known as the catecholamines).

Phenylalanine uses the same active transport channel as tryptophan to cross the blood-brain barrier, and, in large quantities, interferes with the production of serotonin.

D-phenylalanine (DPA) either as a single enantiomer or as a component of the racemic mixture is available through conventional organic synthesis. It does not participate in protein biosynthesis although it is found in proteins, in small amounts, particularly aged proteins and food proteins that have been processed. The biological functions of D-amino acids remain unclear. Some D-amino acids, such as D-phenylalanine, may have pharmacological activity.

DL-Phenylalanine is marketed as a nutritional supplement for its putative analgesic and antidepressant activities. The putative analgesic activity of DL-phenylalanine may be explained by the possible blockage by D-phenylalanine of enkephalin degradation by the enzyme carboxypeptidase A. The mechanism of DL-phenylalanine's putative antidepressant activity may be accounted for by the precursor role of L-phenylalanine in the synthesis of the neurotransmitters norepinephrine and dopamine. Elevated brain norepinephrine and dopamine levels are thought to be associated with antidepressant effects. D-phenylalanine is absorbed from the small intestine, following ingestion, and transported to the liver via the portal circulation. A fraction of D-phenylalanine appears to be converted to L-phenylalanine. D-phenylalanine is distributed to the various tissues of the body via the systemic circulation. D-phenylalanine appears to cross the blood-brain barrier with less efficiency than L-phenylalanine. A fraction of an ingested dose of D-phenylalanine is excreted in the urine.

The genetic disorder phenylketonuria (PKU) is the inability to metabolize phenylalanine. Individuals with this disorder are known as “phenylketonurics” and must abstain from consumption of phenylalanine. This dietary restriction also applies to pregnant women with hyperphenylalanine (high levels of phenylalanine in blood) because they do not properly metabolize the amino acid phenylalanine. Persons suffering from PKU must monitor their intake of protein to control the buildup of phenylalanine as their bodies convert protein into its component amino acids.

A related issue is the compound present in many sugarless gums and mints, snack foods, sugarless soft drinks (such as diet sodas including CocaCola Zero, some forms of Lipton Tea, Clear Splash flavored water), and a number of other low calorie food products. The artificial sweetener aspartame, sold under the names “Equal” and “NutraSweet”, is an ester that is hydrolyzed in the body to give phenylalanine, aspartic acid, and methanol (wood alcohol). The breakdown problems phenylketonurics have with protein and the attendant build up of phenylalanine in the body also occurs with the ingestion of aspartame, although to a lesser degree. Accordingly, all products in the U.S. and Canada that contain aspartame must be labeled: “Phenylketonurics: Contains phenylalanine.” In the UK, foods containing aspartame must carry ingredients panels that refer to the presence of ‘aspartame or E951’, and they must be labeled with a warning “Contains a source of phenylalanine”. These warnings are specifically placed to aid individuals who suffer from PKU so that they can avoid such foods.

Interestingly, the macaque genome was recently sequenced and it was found that macaques naturally have a mutation that is found in humans who have PKU.

Phenylalanine is not known to have any aphrodisiac effects that have been proven. It does convert to tyrosine which is a precursor to dopamine. This can help people who are depressed. If people are depressed they are likely not interested in sex and taking phenylalanine could aid in getting them in the mood. Since tyrosine converts to L-Dopa, which is further converted into dopamine, norepinephrine (noradrenaline), and epinephrine (adrenaline), it can also aid in lifting depression. Clearly a shot of adrenaline can lighten your mood. However, adrenaline itself is not considered an aphrodisiac.

Omega-3 fatty acids are a family of polyunsaturated fatty acids, which have in common a carbon-carbon double bond in the ω-3 position.

Important nutritional essential omega-3 fatty acids are: α-linolenic acid (ALA), eicosapentaenoic acid (EPA), and docosahexaenoic acid (DHA). Other omega-3 fatty acids include stearidonic acid, stearidonic acid, and docosapentaenoic acid. The human body cannot synthesize omega-3 fatty acids de novo (synthesis of complex molecules from simple molecules), but it can form 20- and 22-carbon unsaturated omega-3 fatty acids from the eighteen-carbon omega-3 fatty acid, α-linolenic acid. These conversions occur competitively with omega-6 fatty acids, which are essential closely related chemical analogues that are derived from linoleic acid. Both the omega-3 α-linolenic acid and omega-6 linoleic acid are essential nutrients, which must be obtained from food. Synthesis of the longer omega-3 fatty acids from linolenic acid, within the body is competitively slowed by the omega-6 analogues. Thus, accumulation of long-chain omega-3 fatty acids in tissues is more effective when they are obtained directly from food or when competing amounts of omega-6 analogs do not greatly exceed the amounts of omega-3.

Omega-6 fatty acids are fatty acids where the term “omega-6” signifies that the first double bond in the carbon backbone of the fatty acid, occurs in the omega minus 6 position; that is, the sixth carbon from the end of the fatty acid. The biological effects of the ω-6 fatty acids are largely mediated by their interactions with the ω-3 fatty acids.

Linoleic acid, the shortest chain omega-6 fatty acid is an essential fatty acid. Arachidonic acid is a physiologically significant n-6 fatty acid and is the precursor for prostaglandins and other physiologically active molecules.

Some medical research has suggested that excessive levels of omega-6 acids, relative to omega-3 fatty acids, may increase the probability of a number of diseases and depression. Modern Western diets typically have ratios of omega-6 to omega-3 in excess of 10 to 1, some as high as 30 to 1. The optimal ratio is thought to be 4 to 1 or lower.

Omega-9 fatty acids are a class of unsaturated fatty acids which have a C═C double bond in the ω-9 position. Some ω-9's are common components of animal fat and vegetable oil.

Two commercially important ω-9 fatty acids are:
Oleic acid, which is a main component of olive oil and other monounsaturated fats.
Erucic acid, which is found in rapeseed, wallflower seed, and mustard seed. Rapeseed with high erucic acid content is grown for commercial use in paintings and coatings as a drying oil.

Unlike ω-3 and ω-6 fatty acids, ω-9 fatty acids are not classed as essential fatty acids (EFA). This is both because they can be created by the human body from unsaturated fat and are therefore not essential in the diet, and because the lack of an ω-6 double bond keeps them from participating in the reactions that form the eicosanoids.

Under severe conditions of EFA deprivation, mammals will elongate and desaturate oleic acid to make mead acid, (20:3 ω-9). (Lipomics). This also occurs to a lesser extent in vegetarians and semi-vegetarians. (Phinney, 1990)

The fatty acids are not known to have any aphrodisiac effects on humans.

Hemp oil is from the seed of the hemp plant that contains between 25-35% oil by weight, which is high in essential fatty acids. Cold-pressed, unrefined hemp oil is light green, with a nutty, grassy flavor.

Refined hemp oil is clear with little flavor. It is widely used in body care products, lubricants, paints and industrial uses. Antimicrobial properties make it a useful ingredient for soaps, shampoos and detergents. The oil is of high nutritional value because its 3:1 ratio of omega-6 to omega-3 essential fatty acids matches the balance required by the human body. It has also received attention in recent years as a possible source of bio-diesel. There are a number of organizations that promote the production and use of hemp oil.

Hemp oil is deliberately manufactured to contain no significant amounts of THC and is therefore not a psychoactive drug.

Hemp oil is not known to have any aphrodisiac effects on humans.

Chocolate—The word “chocolate” comes from the Aztecs of Mexico, and is derived from the Nahuatl word xocolat, which is a combination of the words, xocolli, meaning “bitter”, and atl, which is “water”. The Aztecs associated chocolate with Xochiquetzal, the goddess of fertility. Chocolate is also associated with the Mayan god of fertility. Mexican philologist Ignacio Davila Garibi, proposed that “Spaniards had coined the word by taking the Mayan word chocol and then replacing the Mayan term for water, haa, with the Aztec one, atl.” However, it is more likely that the Aztecs themselves coined the term, having long adopted into Nahuatl the Mayan word for the “cacao” bean; the Spanish had little contact with the Mayans before Cortés' early reports to the Spanish King of the beverage known as xocolatl. However, Micheal D. Coe, professor Emeritus of Anthropology and Curator Emeritus in the Peabody Museum of Natural History at Yale University, and coauthor of the book The True History of Chocolate, argues that the word xocolatl appears in “no truly early source in the Nahuatl language or in Aztec culture.”

Chocolate has been used solely as a drink for nearly all of its history. The earliest record of using chocolate pre-dates the Mayans. Chocolate residue has been found in pottery dating to 1100 BC from Honduras, and 600-400 BC from Belize. The chocolate residue found in an early classic ancient Mayan pot in Río Azul, northern Guatemala, suggests that Mayans were drinking chocolate around 400 A.D. In the New World, chocolate was consumed in a bitter, spicy drink called xocoatl, and was often flavored with vanilla, chile pepper, and achiote, (which is known today as annatto). Xocoatl was believed to fight fatigue, a belief that is probably attributable to the theobromine content. Other chocolate drinks combined it with such edibles as maize starch paste (which acts as an emulsifier and thickener), various fruits, and honey. In 1689 noted physician and collector Hans Sloane, developed a milk chocolate drink in Jamaica, which was initially used by apothecaries, but later sold by the Cadbury brothers (http://www.nhm.ac.uk/research-curation/projects/sloane-herbarium/hanssloane.htm).

Chocolate was also an important luxury good throughout pre-Columbian Mesoamerica, and cacao beans were often used as currency. For example, the Aztecs used a system in which one turkey cost one hundred cacao beans and one avocado was worth three beans.

Romantic lore commonly identifies chocolate as an aphrodisiac. The reputed aphrodisiac qualities of chocolate are most often associated with the simple sensual pleasure of its consumption. More recently, suggestion has been made that serotonin and other chemicals found in chocolate, most notably phenethylamine, can act as mild sexual stimulants. While there is no firm proof that chocolate is indeed an aphrodisiac, giving a gift of chocolate to one's sweetheart is a familiar courtship ritual.

In the early 1980s, chemistry of love researcher Michael Libowitz, author of the popular 1983 book The Chemistry of Love, remarked to reporters that “chocolate was loaded with phenethylamine (PEA)” (Liebowitz, Michael, R. (1983). The Chemistry of Love. Boston: Little, Brown, & Co.). This became the focus for an article in The New York Times, which was then taken up by the wire services, then by magazine free-lancers, and evolved into the now eponymous “chocolate theory of love”. However, phenethylamine is rapidly metabolized by the enzyme MAO-B, preventing significant concentrations from reaching the brain, thus no psychoactive effect is achieved. MAO, Monoamine oxidases, (singular abbreviation MAO) are enzymes that catalyze the oxidation of monoamines. They are found bound to the outer membrane of mitochondria in most cell types in the body. The enzyme was discovered by Mary Hare in the liver, and received the name of tyramine oxidase. Thus the abundance of MAO in the body, prevents significant concentrations of PEA from reaching the brain.

Numerous approaches have been taken in attempts to treat impotence. These approaches include the use of external or internally implanted penile prosthesis. (See, e.g., U.S. Pat. No. 5,065,744, to Zumanowsky). A variety of drugs and methods for administering drugs have also been used in attempts to treat impotence. For example, U.S. Pat. No. 3,943,246 to Sturmer addresses treatment of impotence in men by buccal and peroral administration of daily doses of 300-1500 international units (I.U.) of oxytocin or daily divided doses of 150-250 I.U. of desamino-oxytocin. The patent discloses the buccal administration of 100 I.U. three times a day for 14 days, resulting in improvement of impotentia erectionis in 12 of the 16 patients treated.

U.S. Pat. No. 4,530,920 to Nestor et al. discloses that the administration of nonapeptide and decapeptide analogs of luteinizing hormone releasing hormone (Gonadotropin Releasing Hormone LHRH) agonists may be useful in the induction or enhancement of sexual behavior or therapy for impotence or frigidity. Nestor et al. suggest numerous routes of administration of the analogs including buccal, sublingual, oral, parenteral (including subcutaneous, intramuscular, and intravenous administration), rectal, vaginal, and others.

U.S. Pat. No. 4,139,617 to Grunwell et al. suggests buccal and other routes of administration of 19-oxygenated-androst-5-enes for the endocrine mediated enhancement of the libido in humans.

U.S. Pat. No. 4,863,911 to Anderson et al. discloses methods for treating sexual dysfunction in mammals using a biooxidizable, blood-brain barrier penetrating estrogen derivative. One of the purported objects of the Anderson et al. invention is the treatment of “psychological impotence” in males. Test results showed that the drugs used in the study stimulated mounting behavior, intromission, and mount latency in castrated rats.

A number of publications have proposed the use of various vasodilators for the treatment of impotence in males. Attempts to utilize vasodilators for the treatment of impotence were prompted by the fact that a significant percentage of cases of impotence were noted to be vasculogenic, i.e. resulting from vascular insufficiency.

Voss et al., U.S. Pat. No. 4,801,587, issued Jan. 31, 1989, discloses the use of an ointment containing a vasodilator and a carrier agent for topical application to the penis of impotent men. The Voss et al. patent also describes application of such an ointment into the urethra of the penis using a catheter as well as a multi-step regimen for applying a vasodilator to the skin of the penis. In addition, Voss et al. proposes the surgical removal of a portion of the fibrous sheath surrounding the corpora cavernosum, thereby facilitating the penetration of a vasodilator-containing ointment into the corpora cavernosum. Vasodilators disclosed for use by Voss et al. include papaverine, hydralazine, sodium nitroprusside, phenoxybenzamine, and phentolamine. The Voss et al. patent, however, provides no information regarding the actual efficacy of the treatments proposed or the nature of the response to such treatments.

U.S. Pat. No. 4,127,118 to Latorre describes treating male impotence by direct injection of the vasodilating drugs into the corpus cavernosum and the corpus spongiosum of the penis using a syringe and one or more hypodermic needles. More particularly, the Latorre patent proposes the intracavernosal and intraspongiosal injection of sympathomimetic amines such as nylidrin hydrochloride, adrenergic blocking agents such as tolazoline hydrochloride, and direct acting vasodilators such as isoxsuprine hydrochloride and nicotinyl alcohol.

Brindley, G. S. (Br. J. Pharmac. 87:495-500, 1986) disclosed that, when injected directly into the corpus cavernosum using a hypodermic needle, certain smooth muscle relaxing drugs including phenoxybenzamine, phentolamine, thymoxamine, imipramine, verapamil, papaverine, and naftidrofuryl caused erection. This study noted that injection of an “appropriate dose of phenoxybenzamine or papaverine is followed by an unrelenting erection lasting for hours.” Injection of the other drugs studied induced erections lasting from about 11 minutes to about 6.5 hours.

Zorgniotti et al., J. Urol. 133:39-41 (1985) demonstrated that the intracavernosal injection of a combination of papaverine and phentolamine could result in an erection in otherwise impotent men. Similarly, Althof et al. J. Sex Marital Ther. 17(2): 101-112 (1991) reported that intracavernosal injection of papaverine hydrochloride and phentolamine mesylate resulted in improved erectile ability in about 84% of patients injected. However, in that study the dropout rate was 57%, fibrotic nodules developed in 26% of the patients, 30% of the patients developed abnormal liver function values, and bruising occurred in 19% of the patients.

Other studies describing intracavernosal injection of drugs using hypodermic needles for the treatment of impotence include: Brindley, J. Physiol. 342:24P (1983); Brindley, Br. J. Psychiatr. 143:312-337 (1983); Virag, Lancet ii:978 (1982); and Virag, et al., Angiology 35:79-87 (1984).

While intracavernosal injection may be useful for inducing erections in impotent men, the technique has numerous drawbacks. Obvious drawbacks include pain, risk of infection, inconvenience and interference with the spontaneity of the sex act. Priapism (prolonged and other painful erection) also appears to be a potential problem when using injection methods. See, e.g. Brindley, (1986). Another problem arising in some cases of intracavernosal injection involves the formation of fibrotic lesions in the penis. See, e.g., Corriere, et al., J. Urol. 140:615-617 (1988) and Larsen, et al., J. Urol. 137:292-293 (1987).

Phentolamine, which has been shown to have the potential to induce erection when injected intracavernosally, has also been the subject of oral administration to test its effects in men having non-specific erectile insufficiency (Gwinup, Ann. Int. Med. Jul. 15, 1988, pp. 162-163). In that study, 16 patients ingested either a placebo or a 50 mg orally administered dose of phentolamine. Eleven of the 16 patients (including three placebo-treated patients) became tumescent, became more responsive to sexual stimulation, and were able to achieve an erection sufficient for vaginal penetration after waiting 1.5 hours to attempt intercourse.

Sonda et al. J. Sex & Marital Ther. 16(1): 15-21 (year) reported that yohimbine ingestion resulted in subjective improvement in erectile ability in 38% of impotent men treated, but only 5% of the treated patients reported complete satisfaction.

Zorgniotti et al, PCT/US94/09048, describes the transmucosal administration of a variety of vasodilators including phentolamine mesylate for modulating the human sexual response.

U.S. Pat. No. 4,885,173, to Stanley et al., discloses methods of administering drugs having cardiovascular or renal vascular activity through use of a lollipop assertedly facilitating drug absorption through the mucosal tissues of the mouth, pharynx, and esophagus. The Stanley et al. patent discloses that a large number of lollipop-administered drugs may improve cardiovascular function including drugs exhibiting direct vasodilating effects, including calcium channel blockers, beta-adrenergic blocking agents, serotonin receptor blocking agents, angina blocking agents, other anti-hypertensive agents, cardiac stimulating agents, and agents which improve renal vascular function.

U.S. Pat. No. 5,059,603 to Rubin describes the topical administration to the penis of isoxsuprine and caffeine, and nitroglycerine and caffeine along with suitable carrier compounds for the treatment of impotence.

U.S. Pat. No. 5,902,593 to Kent, et al., discloses a topically applied aphrodisiac dispersed in a manually applied vehicle, which substantially increases tissue sensation. The principal ingredient is benzalkonium chloride in a water-soluble gel, which includes sorbitol, glycerin, hydroxethylcellulose and propylene glycol.

U.S. Pat. No. 5,281,423 to Reilly discloses a method of heightening sexual desire of an adult human female comprising administering to an adult human female in need of said treatment, an effective amount of hydriodic acid syrup, comprising hydriodic acid, water and dextrose.

U.S. Pat. No. 3,943,246 to Sturmer discloses a method of treating impotency in a human male in need of said treatment, which comprises administering oxytocin at a dosage of 300 to 1500 I.U. daily.

The above issued patents comprise means, which are either considered the ingestion of a drug orally or by other means. The drugs are not a good solution since it is highly probable that prolonged use will result in side effects that are unwanted.

Natural remedies for which patents have been issued include:

U.S. Pat. No. 6,803,060 to Reyes discloses a composition for boosting libido of an individual, said composition consisting essentially of an effective amount 667 mg Tribulus, 427 mg Muria Puama, 352 mg, Catuba Bark, 127 mg L-Arginine, 60 mg Avena Sativa, and 37 IU Vitamin E.

Reyes further discloses a spray composition for boosting libido of an individual, said composition consisting essentially of 35 mg Tribulus terrestris, 30 mg Epimedium sagattatium, 10 mg Muria Puama, 10 mg Serenoa reopens, 10 mg Chrysin, and 4 mg 5-Androstenediol.

U.S. Pat. No. 6,093,421 to DeLuca et al. discloses a process of increasing testosterone levels in a man comprising orally administering to a man in need of such a treatment, an effective amount of a composition containing Maca and antler.

### DETAILED DESCRIPTION OF THE INVENTION

The proposed invention is not a vasodilator. It is a combination of specific amino acids with an additive catalyst substance which promotes the amino acids to stimulate the glands in the brain generating a psychotropic effect causing the body to generate its own unique chemistry specific to the augmentation and enhancement of sexual function. The effect is not gender specific but works on male as well as female humans. In the absence of the catalyst substance the amino acids simply will not have the effects reported, thus the invention would be inoperative. The catalyst substance by itself has no aphrodisiac properties. It is therefore unobvious to combine it with said amino acids. The unexpected result can be measured in the following way. If the amino acids are used by an individual the aphrodisiac effects are negligible and or absent entirely. When the amino acids are combined with the catalyst, powerful and profound aphrodisiac effects are experienced. The amino acids employed are Tryptophan and Phenylalanine. The catalyst substance is a blend of omega oils, in particular hemp seed oil.

First of all tryptophan is a substance that helps you sleep. Phenylalanine is a substance that helps adrenaline production and is a stimulant. It is unobvious to combine them for any reason since it appears from the conventional wisdom that they would be achieving opposite results. One would be attempting to put you asleep, while the other would be trying to keep you awake. It is an unobvious and unexpected discovery of the invention that their combination with effective amounts of omega oils comprises a powerful aphrodisiac.

In one embodiment of the proposed invention, the amino acids tryptophan and phenylalanine are combined with an effective amount of omega oils for the enhancement of libido.

In one embodiment of the proposed invention, the amino acids tryptophan and phenylalanine are combined with an effective amount of omega oils for the enhancement of libido, the amount of phenylalanine being greater than the amount of tryptophan by weight.

In one embodiment of the proposed invention, the amino acids tryptophan and phenylalanine are combined with an effective amount of omega oils for the enhancement of libido, the amount of phenylalanine being greater than the amount of tryptophan by weight. The amount of amino acids tryptophan and phenylalanine, and the omega oils being adjusted to put the Ph of the fluid at room temperature in the neighborhood of 6.4-7.6.

In one embodiment of the proposed invention, the amino acids tryptophan and phenylalanine are combined with an effective amount of omega oils for the enhancement of libido. The phenylalanine is dissolved in the omega oils. The tryptophan is dissolved in water. The two fluids are then combined into a single mix wherein the Ph is adjusted by altering the amount of each amino acid to bring the Ph within the ranges 6.4-7.6.

In one embodiment of the proposed invention, the amino acids tryptophan and phenylalanine are combined with an effective amount of omega oils for the enhancement of libido. Further included is an effective amount of chocolate.

In one embodiment of the proposed invention, the amino acids tryptophan and phenylalanine are combined with an effective amount of omega oils for the enhancement of libido. Further included is an effective amount of chocolate, the ratio of chocolate to the amino acid oil blend being specific to optimize taste as well as aphrodisiac effects.

What we claim is:

1. A method of enhancing the libido in an adult human comprising the administering of a composition, comprising an effective amount of the amino acids tryptophan and phenylalanine and an effective amount of omega oils.

2. The method claim one wherein the ratio of the amount of phenylalanine to the amount of tryptophan is greater than one.

3. The method of claim one wherein the ratio of the amount of phenylalanine to the amount of tryptophan is greater than or equal to two to one.

4. The method of claim one wherein the amount of amino acid added to a given volume of omega oils is such that the pH is between 6.8 and 7.2.

5. The method of claim one wherein said amount of omega oils are in the form of hemp seed oil.

6. The method of claim one wherein said amount of omega oils are in the form of flax seed oil.

7. The method of claim one wherein said amount of phenylalanine is dissolved in omega oil and said amount of tryptophan is dissolved in water and said solutes are mixed together wherein said amounts of phenylalanine and tryptophan are adjusted so as to provide a pH between 6.4 and 7.6.

8. The method of claim one wherein said amount of phenylalanine is dissolved in omega oil and said amount of tryptophan is dissolved in water and said solutes are mixed together wherein said amounts of phenylalanine and tryptophan are adjusted so as to provide a pH between 6.8 and 7.2.

9. The method of claim one wherein said amount of phenylalanine is dissolved in omega oil and said amount of tryptophan is dissolved in a water base chocolate syrup and said solutes are mixed together wherein said amounts of phenylalanine and tryptophan are adjusted so as to provide a pH between 6.4 and 7.6, and said amounts of said amino acids are such that for every ounce of chocolate syrup mixture there exists between 250 and 300 mg of phenylalanine and between 125 and 150 mg of tryptophan.

10. The method of claim one wherein said amount of phenylalanine is dissolved in water and said amount of tryptophan is dissolved in a water, said water is used as a base in a water base chocolate syrup and said amounts of phenylalanine and tryptophan are adjusted so as to provide a Ph between 6.4 and 7.6, and said amounts of said amino acids are such that for every ounce of chocolate syrup mixture there exists between 250 and 300 mg of phenylalanine and between 125 and 150 mg of tryptophan.

11. The method of claim one wherein said amount of phenylalanine is dissolved in omega oil and said amount of tryptophan is dissolved in a water base chocolate syrup and said solutes are mixed together wherein said amounts of phenylalanine and tryptophan are adjusted so as to provide a pH between 6.4 and 7.6, and said amounts of said amino acids are such that for every ounce of chocolate syrup mixture there exists between 250 and 300 mg of phenylalanine and between 125 and 150 mg of tryptophan.

12. The method of claim one wherein said amount of phenylalanine is dissolved in omega oil and said amount of tryptophan is dissolved in a water base chocolate syrup and said solutes are mixed together wherein said amounts of phenylalanine and tryptophan are adjusted so as to provide a pH between 6.4 and 7.6.
